(19) United States
(12) Patent Application Publication
(10) Pub. No.: US 2018/0236001 A1
(43) Pub. Date: Aug. 23, 2018

(54) METHODS OF GROWING ACID RESISTANT LAYERS ON TOOTH ENAMEL

(71) Applicant: CAO GROUP, INC., West Jordan, UT (US)

(72) Inventor: Steven D. Jensen, West Jordan, UT (US)

(21) Appl. No.: 15/752,090

(22) PCT Filed: Aug. 11, 2016
(86) PCT No.: PCT/US16/46485

§ 371 Date: Feb. 12, 2018

Related U.S. Application Data
(60) Provisional application No. 62/204,066, filed on Aug. 12, 2015.

(51) Int. Cl. A61K 33/42; A61K 9/00; A61P 1/02

(52) U.S. Cl. CPC A61K 33/42; A61P 1/02; A61K 9/0063

(57) ABSTRACT

A method of reducing tooth decay may include growing one or more acid resistant layers on tooth enamel. The acid resistant layer may include a monofluorophosphate anion. For example the acid resistant layer may include calcium monofluorophosphate. An acid resistant layer may be grown by delivering a two-part compound to the tooth enamel.

### CROSS-REFERENCES TO RELATED APPLICATIONS

This application claims the benefit of PCT Application No. PCT/US2016/046485, filed Aug. 11, 2016, which is a non-provisional of U.S. Provisional Patent Application No. 62/204,066 filed on Aug. 12, 2015, which is hereby incorporated by reference in its entirety.

### TECHNICAL FIELD OF THE INVENTION

The present invention discloses methods of growing acid resistant layers on tooth enamel

### SUMMARY

Embodiments of the present invention provide methods of growing acid resistant layers on tooth enamel.

### BRIEF DESCRIPTION OF THE DRAWINGS

In order to describe the manner in which the above-recited and other advantages and features of the invention can be obtained, a more particular description of the invention briefly described above will be rendered by reference to specific example embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings.

FIG. 1 depicts a layer grown on the enamel surface of a tooth according to an embodiment of the present invention.

### DETAILED DESCRIPTION OF THE INVENTION

Tooth caries have plagued mankind since time forgotten. It is known that the acidic by-products of bacterium cause tooth caries as they consume residual food that remain on the teeth after eating. (Bacterium mainly produce lactic acid as a by-product) Tooth enamel is comprised of calcium hydroxyphosphate, Ca₅(PO₄)₃0H or hydroxyapatite. Acid ionically dissolves calcium hydroxyphosphate: Ca₅(PO₄)₃0H₍₅₎+H⁺→Ca₅(PO₄)₃⁺+H₂O. There have been many means devised to combat caries throughout history, i.e. brushing teeth, fluoridated water, fluoride toothpaste, floss, etc. These methods work by various means; removing the residual food from the teeth after eating, making the enamel more resistant to acid, diluting the acid by stimulating saliva flow, etc.

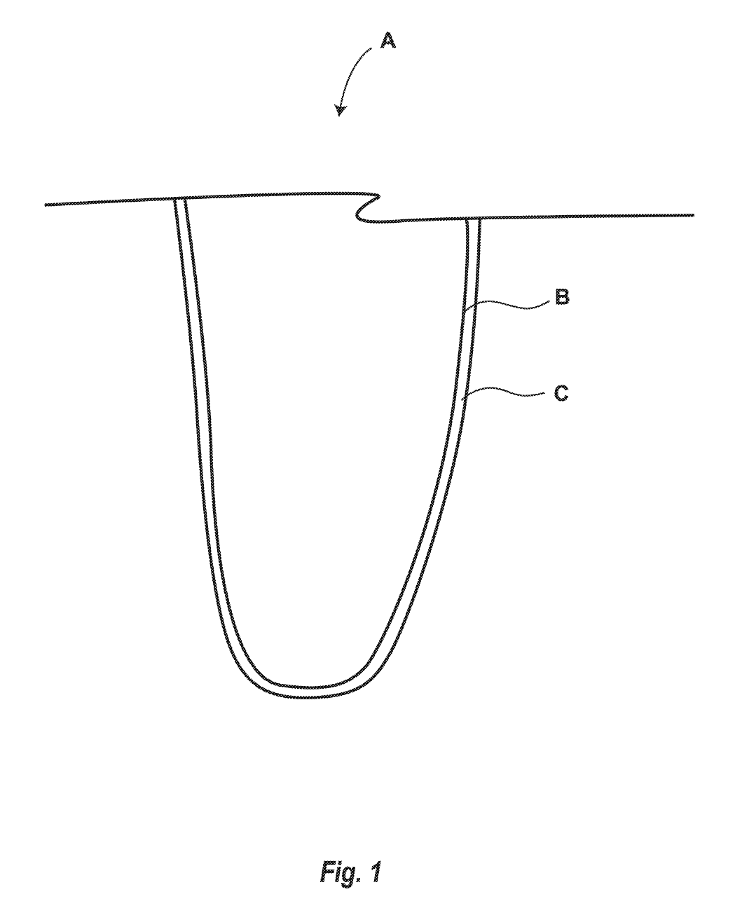

The present invention combats caries by growing an acid resistant layer on tooth structure. FIG. 1 shows a drawing of a portion of a tooth A; wherein the outermost structure is the enamel surface B. Adhered to the enamel is acid resistant layer C. By creating an acid resistant layer C over tooth structure the incidences of acidic erosion produced by the by-products of bacteria is minimized.

The present invention embodies any layer that is grown onto tooth structure that has an acid resistance greater than natural tooth enamel.

One embodiment of the present invention is designed to grow an ionically bound crystalline layer over the surface of tooth structure. The embodiment combines a calcium ion source with an acid resistant anion while within the aqueous/saliva environment of the oral cavity. When combined in solution the ionic compounds begin to precipitate forming a salt; the ions on or at the enamel surface undergo crystalline growth forming a new layer. The process is similar to the re-mineralization processes that occur naturally between saliva and tooth enamel. Saliva contains ionic compounds that grow hydroxyapatite on tooth structure on a constant basis. This embodiment of the present invention is designed to grow a more acid resistant layer over the enamel utilizing the same re-mineralization process as occur naturally within the oral cavity. In this embodiment, we simply incorporate a more acid resistant anion into the newly re-mineralized structure.

Another embodiment of the present invention combines calcium ascorbate with sodium monofluorophosphate (MFP) within the oral cavity, upon aqueous dissolution the combined ionic compounds begin to react to form the more stable salt: calcium monofluorophosphate. The calcium and monofluorophosphate ions on or at the enamel surface undergo crystalline growth forming a new layer. The process is similar to the re-mineralization processes that occurs naturally between saliva and tooth enamel. Saliva contains ionic compounds that grow hydroxyapatite on tooth structure on a constant basis. This embodiment of the present invention is designed to grow a more acid resistant layer over the enamel utilizing the same re-mineralization process as occur naturally within the oral cavity. In this embodiment, we merely incorporate the more acid resistant monofluorophosphate anion into the newly re-mineralized tooth layer.

These ionic compounds of the present invention can be delivered to the oral cavity by various means such as: a 2-part mouthwash, 2-part mixed gel loaded in trays, a 2-part strip that activates when placed into the oral cavity, a 2-part lozenge system that are simultaneously placed into the mouth, and any other useful delivery system that is capable of delivering the components to the oral cavity.

What is claimed is:

1. A method of reducing tooth decay comprising, growing at least one acid resistant layer on tooth enamel.

2. The method of claim 1, wherein the at least one acid resistant layer comprises a monofluorophosphate anion.

3. The method of claim 1, wherein the at least one acid resistant layer comprises calcium monofluorophosphate.

4. The method of claim 1, wherein growing the at least one acid resistant layer on the tooth enamel comprises delivering a two-part compound to the tooth enamel.

5. The method of claim 4, wherein delivering the two-part compound to the tooth enamel comprises delivering a first part comprising calcium ascorbate and a second part comprising sodium monofluorophosphate.

6. The method of claim 4, wherein delivering the two-part compound to the tooth enamel comprises delivering a two-part mouthwash to the tooth enamel.

7. The method of claim 4, wherein delivering the two-part compound to the tooth enamel comprises delivering a two-part gel to the tooth enamel.

8. The method of claim 7, wherein delivering the two-part gel to the tooth enamel loading the two-part gel into a tray.

9. The method of claim 4, wherein delivering the two-part compound to the tooth enamel comprises applying a strip over the tooth enamel.

10. The method of claim 4, wherein delivering the two-part compound to the tooth enamel comprises dissolving a two-part lozenge.
